SEQUENCE LISTING (59 sequences)

SEQ ID NO: 1 - DNA, 19 bp, Artificial Sequence
  Description of Artificial Sequence Probe
atctggcggatcagtggta

SEQ ID NO: 2 - DNA, 19 bp, Artificial Sequence
  Description of Artificial Sequence Probe
atctggcggttcagtggta

SEQ ID NO: 3 - DNA, 19 bp, Artificial Sequence
  Description of Artificial Sequence Probe
atctggcgggtcagtggta

SEQ ID NO: 4 - DNA, 19 bp, Artificial Sequence
  Description of Artificial Sequence Probe
atctggcggctcagtggta

SEQ ID NO: 5 - DNA, 19 bp, Artificial Sequence
  Description of Artificial Sequence Probe
atttggcggatcagtggta

SEQ ID NO: 6 - DNA, 19 bp, Artificial Sequence
  Description of Artificial Sequence Probe
atttggcggttcagtggta

SEQ ID NO: 7 - DNA, 19 bp, Artificial Sequence
  Description of Artificial Sequence Probe
atttggcgggtcagtggta

SEQ ID NO: 8 - DNA, 19 bp, Artificial Sequence
  Description of Artificial Sequence Probe
atttggcggctcagtggta

SEQ ID NO: 9 - DNA, 19 bp, Artificial Sequence
  Description of Artificial Sequence Probe
  modified_base (3): i
atntggcggatcagtggta

SEQ ID NO: 10 - DNA, 19 bp, Artificial Sequence
  Description of Artificial Sequence Probe
  modified_base (3): i
atntggcggttcagtggta

SEQ ID NO: 11 - DNA, 19 bp, Artificial Sequence
  Description of Artificial Sequence Probe
  modified_base (3): i
atntggcgggtcagtggta

SEQ ID NO: 12 - DNA, 19 bp, Artificial Sequence
  Description of Artificial Sequence Probe
  modified_base (3): i
atntggcggctcagtggta

SEQ ID NO: 13 - DNA, 19 bp, Artificial Sequence
  Description of Artificial Sequence Probe
gttagcacaatatctggcg

SEQ ID NO: 14 - DNA, 19 bp, Artificial Sequence
  Description of Artificial Sequence Probe
gttagcacattatctggcg

SEQ ID NO: 15 - DNA, 19 bp, Artificial Sequence
  Description of Artificial Sequence Probe
gttagcacagtatctggcg

SEQ ID NO: 16 - DNA, 19 bp, Artificial Sequence
  Description of Artificial Sequence Probe
gttagcacactatctggcg

SEQ ID NO: 17 - DNA, 19 bp, Artificial Sequence
  Description of Artificial Sequence Probe
gttagcacaatatttggcg

SEQ ID NO: 18 - DNA, 19 bp, Artificial Sequence
  Description of Artificial Sequence Probe
gttagcacattatttggcg

SEQ ID NO: 19 - DNA, 19 bp, Artificial Sequence
  Description of Artificial Sequence Probe
gttagcacagtatttggcg

SEQ ID NO: 20 - DNA, 19 bp, Artificial Sequence
  Description of Artificial Sequence Probe
gttagcacactatttggcg

SEQ ID NO: 21 - DNA, 19 bp, Artificial Sequence
  Description of Artificial Sequence Probe
gctagcacaatatctggcg

SEQ ID NO: 22 - DNA, 19 bp, Artificial Sequence
  Description of Artificial Sequence Probe
gctagcacattatctggcg

SEQ ID NO: 23 - DNA, 19 bp, Artificial Sequence
  Description of Artificial Sequence Probe
gctagcacagtatctggcg

SEQ ID NO: 24 - DNA, 19 bp, Artificial Sequence
  Description of Artificial Sequence Probe
gctagcacactatctggcg

SEQ ID NO: 25 - DNA, 19 bp, Artificial Sequence
  Description of Artificial Sequence Probe
gctagcacaatatttggcg

SEQ ID NO: 26 - DNA, 19 bp, Artificial Sequence
  Description of Artificial Sequence Probe
gctagcacattatttggcg

SEQ ID NO: 27 - DNA, 19 bp, Artificial Sequence
  Description of Artificial Sequence Probe
gctagcacagtatttggcg

SEQ ID NO: 28 - DNA, 19 bp, Artificial Sequence
  Description of Artificial Sequence Probe
gctagcacactatttggcg

SEQ ID NO: 29 - DNA, 19 bp, Artificial Sequence
  Description of Artificial Sequence Probe
  modified_base (2): i
  modified_base (14): i
gntagcacaatatntggcg

SEQ ID NO: 30 - DNA, 19 bp, Artificial Sequence
  Description of Artificial Sequence Probe
  modified_base (2): i
  modified_base (14): i
gntagcacattatntggcg

SEQ ID NO: 31 - DNA, 19 bp, Artificial Sequence
  Description of Artificial Sequence Probe
  modified_base (2): i
  modified_base (14): i
gntagcacagtatntggcg

SEQ ID NO: 32 - DNA, 19 bp, Artificial Sequence
  Description of Artificial Sequence Probe
  modified_base (2): i
  modified_base (14): i
gntagcacactatntggcg

SEQ ID NO: 33 - DNA, 19 bp, Artificial Sequence
  Description of Artificial Sequence Probe
gttagcacagtatctggcg

SEQ ID NO: 34 - DNA, 19 bp, Artificial Sequence
  Description of Artificial Sequence Probe
gttagcactgtatctggcg

SEQ ID NO: 35 - DNA, 19 bp, Artificial Sequence
  Description of Artificial Sequence Probe
gttagcacggtatctggcg

SEQ ID NO: 36 - DNA, 19 bp, Artificial Sequence
  Description of Artificial Sequence Probe
gttagcaccgtatctggcg

SEQ ID NO: 37 - DNA, 19 bp, Artificial Sequence
  Description of Artificial Sequence Probe
gttagcacagtatttggcg

SEQ ID NO: 38 - DNA, 19 bp, Artificial Sequence
  Description of Artificial Sequence Probe
gttagcactgtatttggcg

SEQ ID NO: 39 - DNA, 19 bp, Artificial Sequence
  Description of Artificial Sequence Probe
gttagcacggtatttggcg

SEQ ID NO: 40 - DNA, 19 bp, Artificial Sequence
  Description of Artificial Sequence Probe
gttagcaccgtatttggcg

SEQ ID NO: 41 - DNA, 19 bp, Artificial Sequence
  Description of Artificial Sequence Probe
gctagcacagtatctggcg

SEQ ID NO: 42 - DNA, 19 bp, Artificial Sequence
  Description of Artificial Sequence Probe
gctagcactgtatctggcg

SEQ ID NO: 43 - DNA, 19 bp, Artificial Sequence
  Description of Artificial Sequence Probe
gctagcacggtatctggcg

SEQ ID NO: 44 - DNA, 19 bp, Artificial Sequence
  Description of Artificial Sequence Probe
gctagcaccgtatctggcg

SEQ ID NO: 45 - DNA, 19 bp, Artificial Sequence
  Description of Artificial Sequence Probe
gctagcacagtatttggcg

SEQ ID NO: 46 - DNA, 19 bp, Artificial Sequence
  Description of Artificial Sequence Probe
gctagcactgtatttggcg

SEQ ID NO: 47 - DNA, 19 bp, Artificial Sequence
  Description of Artificial Sequence Probe
gctagcacggtatttggcg

SEQ ID NO: 48 - DNA, 19 bp, Artificial Sequence
  Description of Artificial Sequence Probe
gctagcaccgtatttggcg

SEQ ID NO: 49 - DNA, 19 bp, Artificial Sequence
  Description of Artificial Sequence Probe
  modified_base (2): i
  modified_base (14): i
gntagcacagtatntggcg

SEQ ID NO: 50 - DNA, 19 bp, Artificial Sequence
  Description of Artificial Sequence Probe
  modified_base (2): i
  modified_base (14): i
gntagcactgtatntggcg

SEQ ID NO: 51 - DNA, 19 bp, Artificial Sequence
  Description of Artificial Sequence Probe
  modified_base (2): i
  modified_base (14): i
gntagcacggtatntggcg

SEQ ID NO: 52 - DNA, 19 bp, Artificial Sequence
  Description of Artificial Sequence Probe
  modified_base (2): i
  modified_base (14): i
gntagcaccgtatntggcg

SEQ ID NO: 53 - DNA, 20 bp, Artificial Sequence
  Description of Artificial Sequence Primer
ccatacctacggcgataccg

SEQ ID NO: 54 - DNA, 20 bp, Artificial Sequence
  Description of Artificial Sequence Primer
gcctgaagccggtacaccgt

SEQ ID NO: 55 - DNA, 18 bp, Artificial Sequence
  Description of Artificial Sequence Synthetic oligonucleotide
tctagacagccactcata

SEQ ID NO: 56 - DNA, 19 bp, Artificial Sequence
  Description of Artificial Sequence Synthetic oligonucleotide
gattggacgagtcaggagc

SEQ ID NO: 57 - DNA, 19 bp, Artificial Sequence
  Description of Artificial Sequence Synthetic oligonucleotide
taatgggtaaataccatcc

SEQ ID NO: 58 - DNA, 20 bp, Artificial Sequence
  Description of Artificial Sequence Primer
acgtactaggcaatgactgg

SEQ ID NO: 59 - DNA, 20 bp, Artificial Sequence
  Description of Artificial Sequence Primer
agagtcgccgtcgatggaac

(19) United States
(12) Patent Application Publication
(10) Pub. No.: US 2008/0096203 A1
(43) Pub. Date: Apr. 24, 2008

(54) METHOD AND DEVICE FOR DETECTING QUINOLONE-RESISTANT ESCHERICHIA COLI

(75) Inventors: Xiaolei Yu, Reutlingen (DE); Milorad Susa, Stuttgart (DE); Cornelius Knabbe, Stuttgart (DE); Rolf Schmid, Stuttgart (DE); Till T. Bachmann, Stuttgart (DE)

Correspondence Address: FOLEY AND LARDNER LLP;SUITE 500, 3000 K STREET NW, WASHINGTON, DC, 20007, US

(73) Assignee: Eppendorf AG

(21) Appl. No.: 11/767,145

(22) Filed: Jun. 22, 2007

Related U.S. Application Data
(62) Division of application No. 10/671,883, filed on Sep. 29, 2003, now abandoned.

(51) Int. Cl. C12Q 1/68

(52) U.S. Cl. 435/6

(57) ABSTRACT

The present invention pertains to a method for detecting quinolone-resistant Escherichia coli strains in a biological sample. The present invention also relates to a kit adapted to perform the inventive method.

This application is a divisional of U.S. application Ser. No. 10/671,883, filed Sep. 29, 2003, the disclosure of which is herein incorporated by reference in its entirety.

### FIELD OF THE INVENTION

The present invention pertains to a method for detecting quinolone-resistant Escherichia coli strains in a biological sample material. The present invention also relates to a kit adapted to perform the present method.

### BACKGROUND OF INVENTION

Bacterial infections are generally treated with antibiotics, among which quinolones have proven to be one of the most highly potent agents for use in human. In the past, fluoroquinolones have been widely used as broad spectrum antimicrobial agents in clinical medicine with the result that bacteria have developed resistance against this agent.

One of the most concerned species of bacteria to be treated with quinolones is E. coli which causes a number of infections, primarily in and around artificial or natural openings of the body, such as lesions in the skin or the urinary tract. Particularly, experience in and information about the treatment of urinary tract infections shows that 90% of the antibiotics administered are quinolones, while in the meantime about 8% of the E. coli strains have become resistant. Therefore, the ordinary regimen does not apply in a number of cases, which the attending physician will normally recognize only at a later stage of the infection/bacterial growth, with a concurrent destruction of the infested tissue. In addition, quinolone-resistant E. coli may also prove to be a potential threat to neutropenic patients with leukemia, who receive a quinolone as prophylaxis.

In general, the therapeutic or prophylactic use of quinolones without considering possible resistance of the infecting pathogen may lead to treatment failures as well as to an induction of new resistances.

Therefore, there is a need in the art to get information about potential resistances occurring in the bacterial population to be treated.

Up to now the standard methods to determine an antibiotic resistance are based on phenotypic identification, which is time consuming and is in certain cases not sensitive and precise enough.

An approach in the art to cope with these problems focuses on the investigation of polypeptides accounting for the quinolone resistance in pathogenic bacteria. Several analyses have been developed in order to gain such information, for example a single-stranded conformational polymorphism (SSCP) analysis (Ouabdesselam S, Hooper D C, Tankovic J, Soussy C J, Antimicrobial Agents and Chemotherapy 39 (1995), 1667-70), a mismatch amplification mutation assay (MAMA; Qiang Y Z, Qin T, Fu W, Cheng W P, Li Y S, Yi G., J Antimicrob Chemother 49 (2002), 549-52) and a restriction fragment length polymorphism (RFLP) analysis (Hooper D C, Wolfson J S, Ng E Y, Swartz M N., Am J Med 82 (1987), 12-20).

However, all the above methods and assays exhibit a variety of different shortcomings. In particular, with a SSCP only the region of mutation may be detected, but not the exact position of mutation. With the MAMA procedure, only one variant may be determined at a time, or else a cost and work intensive multiplex PCR has to be performed. RFLP detects only the position of the mutation, but not the substitution. In addition, none of the methods accurately predicts whether the bacterial sample exhibits resistance to the agents utilized.

Therefore, a need exists to rapidly and reliably detect the presence of resistant strains of bacteria. Furthermore, such a detection assay should process multiple samples simultaneously and inexpensively.

### SUMMARY OF THE INVENTION

It is, therefore, one object of the present invention to provide a method for detecting the presence of quinolone resistant E. coli strains in a biological sample.

It is also an object of the present invention to provide micro-arrays and kits for use in detecting the presence of quinolone resistant E. coli strains in a biological sample.

In accomplishing these and other objects of the invention, there is provided, in accordance with one aspect of the invention a method for detecting the presence of quinolone resistant E. coli strains in a biological sample, which method comprises the steps (i) obtaining DNA from a biological sample, (ii) optionally amplifying the DNA contained in the sample with primers specific for the target sequence, (iii) contacting the DNA contained in the biological sample or obtained in step (ii) with a micro-array comprising at specific predetermined locations of the array two sets of capture probes, which are derived from the sequence of a gyrA gene of E. coli, and comprise the sequence R₁—(X)—R₂, wherein (a) X designates all permutations of the triplet at amino acid position 83 and 87 of the gyrA polypeptide of E. coli, and wherein (b) R₁ and R₂ are sequences derived from the gyrA gene of E. coli adjacent to the triplet of either position 83 or 87 of the gyrA polypeptide and comprising of from about 5 to 20 nucleotides, under conditions allowing hybridization of complementary strands, and (iv) determining, at which location on the array binding occurs, wherein a change in the nucleic acid at the said positions resulting in a change of an amino acid is indicative of the development of a resistance against quinolones. In one embodiment, the change in the nucleic acid sequence results in an amino acid change of the gyrA polypeptide to leucine at position 83 and/or asparagine or tyrosine at position 87.

The invention also provides a micro-array containing at specific predetermined locations of the array two sets of capture probes, derived from the sequence of a gyrA gene of E. coli, comprising the sequence R₁—(X)—R₂, wherein (a) X designates all permutations of the triplet at amino acid position 83 and 87 of the gyrA polypeptide of E. coli and (b) R₁ and R₂ are sequences derived from the gyrA gene of E. coli adjacent to the triplet of either position 83 or 87 of the gyrA polypeptide and comprising of from about 5 to 20 nucleotides.

In another embodiment, there is provided a kit for detecting the presence or absence of a quinolone resistant E. coli strain in a biological sample, containing a micro-array containing at specific predetermined locations of the array two sets of capture probes, derived from the sequence of a gyrA gene of E. coli, comprising the sequence R₁—(X)—R₂, wherein (a) X designates all permutations of the triplet at amino acid position 83 and 87 of the gyrA polypeptide of E. coli and (b) R₁ and R₂ are sequences derived from the gyrA gene of E. coli adjacent to the triplet of either position 83 or 87 of the gyrA polypeptide and comprising of from about 5 to 20 nucleotides, and optionally buffers and reagents.

Other objects, features and advantages of the present invention will become apparent from the following detailed description. The detailed description and specific examples, while indicating preferred embodiments, are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE FIGURES

FIGS. 1A-D show the results of a hybridization of clinical isolates with labeled target DNA on a micro-array.

### DETAILED DESCRIPTION OF THE INVENTION

In the studies leading to the present invention a number of clinical isolates of Et coil known to be quinolone resistant have been investigated, while it has been surprisingly noted that in contrast to the quinolone sensitive strain, all of the resistant strains exhibited mutations in the gyrA polypeptide in at least one of amino acid positions 83 and 87. This focus on these two amino acid positions in resistant strains has been confirmed by additional studies so that the present invention is essentially based on the finding that in order to detect a quinolone resistance in E. coli, it is sufficient to provide data about these two positions in the gyrA polypeptide of E. coli, only.

Without wishing to be bound to any theory, it is presently believed that even though these two positions are not the sole mutations occurring in the gyrA polypeptide of quinolone resistant strains, they seem to be mainly involved in the development of resistance due to a folding of the resulting polypeptide preventing interaction with quinolones.

Another gene of interest that conveys quinolone resistance is topoisomerase IV. Of particular interest is subunit A, which is encoded by the parC gene. In this gene, three amino acid positions, 80, 84 and 87, are proposed as locations for the detection of quinolone resistance.

### DEFINITIONS

In the present description the following definitions apply:

The terms “micro-array” and “array of oligonucleotides”, which are used interchangeably in the present invention, refer to a multiplicity of different nucleotide sequences attached or positioned on one or more solid supports where, when there is a multiplicity of supports, each support bears a multiplicity of nucleotide sequences. Both terms may refer to the entire collection of nucleotides on the support(s) or to a subset thereof. In one embodiment, the nucleotide sequence is attached through a single terminal covalent bond. The support is generally composed of a solid surface which may be selected from the group consisting of glasses, electronic devices, silicon supports, silica, metal or mixtures thereof prepared in format selected from the group of slides, discs, gel layers and/or beads.

As used in present invention, the term “probe” or “capture probe” in the sense of the present invention is defined as a nucleotide sequence representing specific parts of the gyrA gene or parC gene, respectively, of E. coli covering amino acid positions 83 and 87 (gyrA) or 80, 84 or 87 of parC, respectively. The sequences have different lengths, e.g. between about 10 and 43 nucleotides, and are either chemically synthesized in situ on the surface of the support or laid down thereon. They are capable of binding to a target nucleic acid of complementary sequence through one or more types of chemical bonds, usually through complementary base pairing, usually through hydrogen bond formation. As used herein, a nucleotide probe may include natural (i.e. A, G, C, or T) or modified bases (7-deazaguanosine, inosine, etc.). In addition, the bases in an oligonucleotide probe may be joined by a linkage other than a phosphodiester bond, such as e.g. peptide bonds, so long as it does not interfere with hybridization.

The term “target nucleic acid” refers to a nucleic acid, to which the nucleotide probe specifically hybridizes.

The term “gyrA gene” as used in the present application comprises the gyrA gene of E. coli and its variants due to mutations and changes in different strains.

The term “parC gene” as used in the present application comprises the parC gene sequence of E. coli and its variants due to mutations and changes in different strains.

The term “nucleotide sequence” as used herein refers to oligonucleotide(s), polynucleotide(s) and the like including analogous species wherein the sugar-phosphate backbone is modified and/or replaced, provided that its hybridization properties are not destroyed.

The phrase “hybridizing specifically to” refers to the binding, duplexing or hybridizing of a molecule substantially to or only to a particular nucleotide sequence or sequences under stringent conditions when that sequence is present in a complex mixture of DNA or fragments thereof.

The terms “background” or “background signal intensity” refers to hybridization signals resulting from non-specific binding, or other interactions, between the labeled target nucleic acids and components of the nucleotide array (e.g., the nucleotide probes, control probes, the array substrate, etc.).

### DESCRIPTION

In order to perform the present method, a DNA from a biological sample is obtained in a first step from an individual to be treated or deemed to harbor a resistant strain. The biological sample/material may be any material supposed to contain a pathogenic E. coli, such as tissue from an area of a lesion, blood, or body secretions, such as sputum or urine. For some applications, it may be appropriate to transfer the biological sample into a medium suitable for the growth of E. coli, e.g. on LB agar plates.

The DNA contained in the biological sample may liberated from the E. coli cells or isolated according to techniques well known in the art, e.g. via QIAprep® Spin Miniprep Kit protocol (Qiagen, Hilden, Germany). Alternative appropriate methods for obtaining DNA may be chosen, depending on the specific starting material. Such an isolation step assists in preventing the development of extensive background signals during the hybridization step, in case no other selection step is applied.

In one embodiment, the DNA contained in the biological sample or isolated therefrom may be amplified via a polymerase chain reaction (PCR) using one or more primers, which provides the advantage of augmenting the specific material to be investigated only and also to incorporate a selection step. In case of using one primer only, the complementary strand of the DNA of interest will be synthesized. Alternatively, at least two primers are utilized, allowing an exponential amplification of the material to be investigated. According to an alternative embodiment a nested PCR is carried out, wherein 2 pairs of primers are put to use. A first set of primers is selected to amplify a sequence largely around the target sequence. Then, the second pair of primers are used to amplify a sequence lying within the sequence amplified first. Proceeding accordingly gives the inherent advantage that a second selection step is incorporated in the present method, which assists in reducing the background. After the completion of the PCR reaction, the PCR product may be purified if desired.

When using an amplification step, the DNA may at the same time be labeled, e.g. by including in the amplification process nucleotides harboring an appropriate label. Alternatively, a label may be attached to the nucleic acid, including, for example, nick translation or end-labeling by attachment of a nucleic acid linker joining the sample nucleic acid to a label.

Detectable labels suitable for use in the present invention include any composition detectable by spectroscopic, photochemical, biochemical, immunochernical, electrical, optical or chemical means. Useful labels in the present invention include biotin for staining with labeled streptavidin conjugate, magnetic beads (e.g., DynabeadS™), fluorescent dyes (e.g., cyanine dyes, such as Cy5, fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

In order to allow detection of the presence of a single mutation, the target- or probe-DNA should not be too long, since otherwise renaturation in solution, or base-pairing with a capture probe allowing mismatches may occur. The desired length of such a probe DNA should be of from about 10 to 50 nucleotides, preferably 15 to 40, more preferably 15-30, even more preferred 15-25 nucleotides and may be obtained by either selecting the primers during the amplification step accordingly, or by fragmenting the DNA put to use after an amplification step.

The target-/probe-DNA thus obtained is then contacted with the capture probes on the micro-array under conditions allowing hybridization of complementary strands only. In general, since a difference in at least one nucleotide is studied under certain conditions, stringent hybridization conditions are selected, e.g. adjusting the hybridization temperature to be about 1′-5° C. below the calculated thermal melting point (Tₘ) of a the specific sequence at a defined ionic strength and pH. The Tₘ is the temperature (at the defined ionic strength, pH) at which 50% of the probes complementary to the target sequence hybridize to the target sequence at equilibrium. Another possibility to adjust stringent conditions resides in adding destabilizing agents, such as e.g. formamide.

In principle, the capture probes on the micro-array comprise the sequence R₁—(X)—R₂ and are provided in two sets on the array. In one set, the sequences R₁ and R₂, which may be of a length of from about 5 to about 20 nucleotides each, are derived from the sequences of the gyrA gene of E. coli adjacent to the triplet encoding the amino acid at position 83 in the gyrA polypeptide, while in the second set of capture probes the sequences R₁ and R₂, which may exhibit the same length as indicated above, are derived from the sequence of the gyrA gene of E. coli adjacent to the triplet encoding the amino acid at position 87 in the gyrA polypeptide.

In a preferred embodiment the sequences R₁ and R₂ are designed such that known mutations of the gene encoding the gyrA polypeptide around positions 83 and 87, e.g. at positions 85 and 89, are taken into account. Hence, the positions 85 and 89 may also be permutated to cover all potential exchanges at these positions and permit an extremely accurate means to determine a SNP at positions 83 and 87, respectively.

An exemplary set of capture probes (SEQ ID NOS 1-52, respectively, in order of appearance) is shown in table I below.

TABLE I

| | Po- | | | A- |
|---|---|---|---|---|
| | si- | Varia- | | mino |
| Name | tion | tion | Sequence (3′→5′) | Acid |
| E.coli_GyA83A1 | 83 | 85(GTC) | AT GGT GAC TAG | Stop |
| | | | GCG GTC TA | code |
| E.coli_GyA83T1 | 83 | 85(GTC) | AT GGT GAC TTG | Leu |
| | | | GCG GTC TA | |
| E.coli_GyA83G1 | 83 | 85(GTC) | AT GGT GAC TGG | Trp |
| | | | GCG GTC TA | |
| E.coli_GyA83C1 | 83 | 85(GTC) | AT GGT GAC TCG | Ser |
| | | | GCG GTC TA | |
| E.coli_GyA83A2 | 83 | 85(GTT) | AT GGT GAC TAG | Stop |
| | | | GCG GTT TA | code |
| E.coli_GyA83T2 | 83 | 85(GTT) | AT GGT GAC TTG | Leu |
| | | | GCG GTT TA | |
| E.coli_GyA83G2 | 83 | 85(GTT) | AT GGT GAC TGG | Trp |
| | | | GCG GTT TA | |
| E.coli_GyA83C2 | 83 | 85(GTT) | AT GGT GAC TCG | Ser |
| | | | GCG GTT TA | |
| E.coli_GyA83AU | 83 | 85(GTI) | AT GGT GAC TAG | Stop |
| | | | GCG GTI TA | code |
| E.coli_GyA83TU | 83 | 85(GTI) | AT GGT GAC TTG | Leu |
| | | | GCG GTI TA | |
| E.coli_GyA83GU | 83 | 85(GTI) | AT GGT GAC TGG | Trp |
| | | | GCG GTI TA | |
| E.coli_GyA83CU | 83 | 85(GTI) | AT GGT GAC TCG | Ser |
| | | | GCG GTI TA | |
| E.coli_GyA87A1 | 87 | 85(GTC)/ | GCG GTC TAT AAC | Asn |
| | | 89(ATT) | ACG ATT G | |
| E.coli_GyA87T1 | 87 | 85(GTC)/ | GCG GTC TAT TAC | Tyr |
| | | 89(ATT) | ACG ATT G | |
| E.coli_GyA87G1 | 87 | 85(GTC)/ | GCG GTC TAT GAC | Asp |
| | | 89(ATT) | ACG ATT G | |
| E.coli_GyA87C1 | 87 | 85(GTC)/ | GCG GTC TAT CAC | His |
| | | 89(ATT) | ACG ATT G | |
| E.coli_GyA87A2 | 87 | 85(GTT)/ | GCG GTT TAT AAC | Asn |
| | | 89(ATT) | ACG ATT G | |
| E.coli_GyA87T2 | 87 | 85(GTT)/ | GCG GTT TAT TAC | Tyr |
| | | 89(ATT) | ACG ATT G | |
| E.coli_GyA87G2 | 87 | 85(GTT)/ | GCG GTT TAT GAC | Asp |
| | | 89(ATT) | ACG ATT G | |
| E.coli_GyA87C2 | 87 | 85(GTT)/ | GCG GTT TAT CAC | His |
| | | 89(ATT) | ACG ATT G | |
| E.coli_GyA87A3 | 87 | 85(GTC)/ | GCG GTC TAT AAC | Asn |
| | | 89(ATC) | ACG ATC G | |
| E.coli_GyA87T3 | 87 | 85(GTC)/ | GCG GTC TAT TAC | Tyr |
| | | 89(ATC) | ACG ATC G | |
| E.coli_GyA87G3 | 87 | 85(GTC)/ | GCG GTC TAT GAC | Asp |
| | | 89(ATC) | ACG ATC G | |
| E.coli_GyA87C3 | 87 | 85(GTC)/ | GCG GTC TAT CAC | His |
| | | 89(ATC) | ACG ATC G | |
| E.coli_GyA87A4 | 87 | 85(GTC)/ | GCG GTT TAT AAC | Asn |
| | | 89(ATT) | ACG ATC G | |
| E.coli_GyA87T4 | 87 | 85(GTC)/ | GCG GTT TAT TAC | Tyr |
| | | 89(ATT) | ACG ATC G | |
| E.coli_GyA87G4 | 87 | 85(GTC)/ | GCG GTT TAT GAC | Asp |
| | | 89(ATT) | ACG ATC G | |
| E.coli_GyA87C4 | 87 | 85(GTC)/ | GCG GTT TAT CAC | His |
| | | 89(ATT) | ACG ATC G | |
| E.coli_GyA87AU1 | 87 | 85(GTI)/ | GCG GTI TAT AAC | Asn |
| | | 89(ATI) | ACG ATI G | |
| E.coli_GyA87TU1 | 87 | 85(GTI)/ | GCG GTI TAT TAC | Tyr |
| | | 89(ATI) | ACG ATI G | |
| E.coli_GyA87GU1 | 87 | 85(GTI)/ | GCG GTI TAT GAC | Asp |
| | | 89(ATI) | ACG ATI G | |
| E.coli_GyA87CU1 | 87 | 85(GTI)/ | GCG GTI TAT CAC | His |
| | | 89(ATI) | ACG ATI G | |
| E.coli_GyA87A5 | 87 | 85(GTC)/ | GCG GTC TAT GAC | Asn |
| | | 89(ATT) | ACG ATT G | |
| E.coli_GyA87T5 | 87 | 85(GTC)/ | GCG GTC TAT GTC | Tyr |
| | | 89(ATT) | ACG ATT G | |
| E.coli_GyA87G5 | 87 | 85(GTC)/ | GCG GTC TAT GGC | Asp |
| | | 89(ATT) | ACG ATT G | |
| E.coli_GyA87C5 | 87 | 85(GTC)/ | GCG GTC TAT GCC | His |
| | | 89(ATT) | ACG ATT G | |
| E.coli_GyA87A6 | 87 | 85(GTT)/ | GCG GTT TAT GAC | Asn |
| | | 89(ATT) | ACG ATT G | |
| E.coli_GyA87T6 | 87 | 85(GTT)/ | GCG GTT TAT GTC | Tyr |
| | | 89(ATT) | ACG ATT G | |
| E.coli_GyA87G6 | 87 | 85(GTT)/ | GCG GTT TAT GGC | Asp |
| | | 89(ATT) | ACG ATT G | |
| E.coli_GyA87C6 | 87 | 85(GTT)/ | GCG GTT TAT GCC | His |
| | | 89(ATT) | ACG ATT G | |
| E.coli_GyA87A7 | 87 | 85(GTC)/ | GGG GTC TAT GAC | Asn |
| | | 89(ATC) | ACG ATC G | |
| E.coli_GyA87T7 | 87 | 85(GTC)/ | GCG GTC TAT GTC | Tyr |
| | | 89(ATC) | ACG ATC G | |
| E.coli_GyA87G7 | 87 | 85(GTC)/ | GCG GTC TAT GGC | Asp |
| | | 89(ATC) | ACG ATC G | |
| E.coli_GyA87C7 | 87 | 85(GTC)/ | GCG GTC TAT GCC | His |
| | | 89(ATC) | ACG ATC G | |
| E.coli_GyA87A8 | 87 | 85(GTC)/ | GGG GTT TAT GAC | Asn |
| | | 89(ATT) | ACG ATC G | |
| E.coli_GyA87T8 | 87 | 85(GTC)/ | GCG GTT TAT GTC | Tyr |
| | | 89(ATT) | ACG ATC G | |
| E.coli_GyA87G8 | 87 | 85(GTC)/ | GCG GTT TAT GGC | Asp |
| | | 89(ATT) | ACG ATC G | |
| E.coli_GyA87C8 | 87 | 85(GTC)/ | GCG GTT TAT GCC | His |
| | | 89(ATT) | ACG ATC G | |
| E.coli_GyA87AU2 | 87 | 85(GTI)/ | GCG GTI TAT GAC | Asn |
| | | 89(ATI) | ACG ATI G | |
| E.coli_GyA87TU2 | 87 | 85(GTI)/ | GCG GTI TAT GTC | Tyr |
| | | 89(ATI) | ACG ATI G | |
| E.coli_GyA87GU2 | 87 | 85(GTI)/ | GCG GTI TAT GGC | Asp |
| | | 89(ATI) | ACG ATI G | |
| E.coli_GyA87CU2 | 87 | 85(GTI)/ | GCG GTI TAT GCC | His |
| | | 89(ATI) | ACG ATI G | |

Capture probes directed against amino acid position 83 and 87 of GyrA with consideration of nucleotide variations at position 85 and 89.
All the probes are 19mer and with the SNPs position almost in the middle.
Bold letter indicate the SNPs positions and underline letter indicate the positions with variation.
For position 83 two sets of probes (eight probes) and for position 87 eight sets of probes (32 probes) were designed, which four sets were directed against the first position of the triplet code, while the other four sets are directed against the second position of the triplet code.
Both for position 83 and 87 were universal probes (for position 83 one set and for position 87 two sets) designed, which had inosine at the positions with variations.

According to a preferred embodiment the array may contain at least one additional set of capture probes, derived from the parC gene of E. coli. In fact, the topoisomerase IV is the secondary target for quinolone in the case of E. coli. The point mutation of the A subunit of this enzyme, which is encoded by parC gene, is the main cause for the resistance. Three amino acid positions, i.e. residues, 80, 84 and 87, have been chosen as locations for the detection, Frequent mutations at position 80 include Ser to Ile or Arg. Common mutations at position 84 include Glu to Lys or Gly.

As for the set of probes directed to the gyrA mutations also in this set of probes (directed to the parC gene), the capture probes on the micro-array comprise the sequence R₁—(Y)—R₂ and may be provided in either of one or two or three sets on the array. In one embodiment, the inventive micro-array contains, apart from the capture probes directed to the gyrA gene, capture probes directed to the parC gene. The sequences R₁ and R₂, which may be of a length of from about 5 to about 20 nucleotides each, are derived from the sequences of the parC gene of E. coli adjacent to the triplet encoding the amino acid at position 80 in the parC polypeptide. In a second and third set of capture probes, respectively, the sequences R₁ and R₂, which may exhibit the same length as indicated above, are derived from the sequence of the gyrA gene of E. coli adjacent to the triplet encoding the amino acid no. 84 or 87 in the parC polypeptide.

In a next step it will be determined at which location on the array binding occurred, which is generally achieved by detecting the label that has been attached to/incorporated in the target-DNA prior to the hybridization step, or by performing a labelling reaction on the array. So called “direct labels” are detectable labels that are directly attached to or incorporated into the target (sample) nucleic acid prior to hybridization. In contrast, so called “indirect labels” are joined to the hybrid duplex after hybridization. The indirect label may also be attached to a binding moiety that has been attached to the target nucleic acid prior to the hybridization. For a detailed review of methods of labelling nucleic acids and detecting labelled hybridized nucleic acids see Laboratory Techniques in Biochemistry and Molecular Biology, Vol. 24. Hybridization With Nucleic Acid Probes, P, Tijssen, ed. Elsevier, N.Y., (1993)).

Also, the capture probes present on the array may contain a label at their 3′-end. After binding of the target DNA, the DNA/DNA hybrids are then cleaved with a particular enzyme thus releasing the label from those capture probes, where the target DNA had bound. Therefore, in this embodiment the decrease in signal is representative of the presence of a given nucleotide sequence in the gyrA gene.

Means of detecting labeled target nucleic acids hybridized to probes are well-known to those skilled in the art.

Two types of divergent results may be obtained.

On the one hand it may be noted that at the location representing the triplet of the native, i.e. quinolone sensitive strain, binding occurred which will be indicative of a quinolone sensitive strain.

On the other hand it may also be observed that binding occurs on a location representing a triplet different from the native one. In this case, it should be first determined whether the change in the triplet has led to a change in the respective amino acid, either in one or two of the positions, preferably from serine to leucine (at position 83) and/or aspartate to asparagine or tyrosine at position 87.

This step of evaluating whether the mutation has led to a change of an amino acids may also be obviated by spotting only such kind of mutations on the array which also lead to a change of an amino acid (cf. wobble hypothesis). However, proceeding accordingly harbors the disadvantage that in such a case no signal will be obtained for this position, wherein the skilled person has to rely solely on the positive control to be ascertained that the experiment really worked. For this reason, a micro-array harboring all of the possible mutations of the respective triplets in the corresponding sets is preferred for use in the present method.

The present method, therefore, provides a reliable and rapid means for determining, whether or not a given biological sample contains an E. coli strain, having developed resistance against quinolones. Since the assay is easy to carry out an attending physician may quickly obtain the required information and may apply an appropriate regimen

### EXAMPLE

### Micro-Array for Detection Quinolone-Resistant Escherichia coli

1. Biological Material

A total of 29 quinolone-resistant E. coli clinical isolates from four different hospitals in Germany and one quinolone-sensitive clinical isolate were used in this study. These strains have been isolated from urine (n=20), swab (from the lower leg n=1, foot n=1, throat n=2, groin n=1, abscess n=1, unknown n=1) (n=7), secretion (tracheal secretion n=1, bronchial secretion n=1) (n=2) and blood (n=1) of patients. The susceptibility of the strains against quinolone was determined either by using Ciprofloxacin (n 23) alone or by using both Ciprofloxacin and Levofloxacin (n=7). Genomic DNA was isolated using QIAamp DNA Mini Kit (Qiagen) according to protocol provided by the manufacturer.

2. DNA Sequencing and Amplification

The gyrA gene of some isolates was sequenced by amplifying the gene from the isolates using primers that yielded overlapping fragments. The sequencing of 5 isolates gave the preliminary result that apart from a variety of different mutations all of them had a common mutation at position 83 and 87 in the gyrA gene.

In order to verify the initial finding the region in the gyrA gene, a 417 bp long fragment from nucleotide position 119 to 535 around these positions was amplified by using the following primers:

forward primer Gyr_coli_F1 (5′-ccatacctacggcgataccg-3′) (SEQ ID NO: 53), and

reverse primer Gyr_coli_R1 (5′-gcctgaagccggtacaccgt-3′) (SEQ ID NO: 54).

The PCR mixtures (50 μl) included about 80 ng template of genomic DNA of E. coli), 0.4 μM (pmol/L) of each primer, 0.25 mM dNTPs (desoxyribonucleoside-5′-triphosphate), 1.5 mM Mg²⁺ (mmol/L) and 2.5 U Taq Polymerase (Eppendort). The PCRs were performed in a thermocycler (Eppendorf) using following parameters: 94° C. 5 min; 94° C. 1 min, 52° C. 1 min, 72° C. 1 min for 30 cycles; final elongation 72° C. 10 min. The amplified fragment, which was purified using QIAquick PCR purification kit (Qiagen) according to the manual provided by the manufacture, was used for direct sequencing. The sequencing was done using the same primer pairs with big-dye terminator cycle sequencing kit (Applied Biosystem) and Prism™ 377A-DNA-sequencer (Applied Biosystems).

For all the investigated resistant strains it was noted that they exhibited a mutation at positions 83 and 87, which were at position 83 from serine (codon TCG) to leucine (codon TTG) (n=28) and at position 87 from aspartate (codon GAC) to asparagine (codon AAC)(n=27) or to tyrosinie (codon TAC) (n=1) or to glycine (codon GGC) (n=1). It was also noted that these 30 isolates belong to two variants. The one variant (n=27) had at position 85 codon GTT (Val), at position 91 codon CGT (Arg) and at position 100 codon TAC (Tyr). The other variant (n=3) had at position 85 codon GTC (Val), at position 91 codon CGC (Arg) and at position 100 codon TAT (Tyr).

TABLE 2

| Posi- | Posi- | Posi- | Posi- | Posi- | Number | |
|---|---|---|---|---|---|---|
| tion | tion | tion | tion | tion | of | |
| 83 | 85 | 87 | 89 | 100* | Isolate | Phenotype |

| | | | | | | |
|---|---|---|---|---|---|---|
| TCG | GTT | GAC | CGT | TAC | 1 | sensitive |
| (Ser) | | (Asp) | | | | |
| TTG | GTC | AAC | CGC | TAT | 3 | resistant |
| (Leu) | | (Asn) | | | | |
| TTG | GTT | AAC | CGT | TAC | 24 | resistant |
| (Leu) | | (Asn) | | | | |
| TTG | GTT | TAC | CGT | TAC | 1 | resistant |
| (Leu) | | (Tyr) | | | | |
| TCG | GTT | GGC | CGT | TAC | 1 | resistant |
| (Ser) | | (Gly) | | | | |

Bold letter indicate the nucleotide change, which lead to amino acid substitution (mutation) and underline letter indicate nucleotide change, which have no effect on amino acid (variation). Asterisk indicate that the sequences of this position were determined by sequencing;
Genotype of 30 clinical isolates determined using micro-array. The phenotype was determined by using Ciprofloxacin alone or by using both Ciprofloxacin and Levofloxacin.

3. Array Fabrication

The possibility of using a micro-array to enable high through-put analysis was evaluated. Using Microgrid II (Biorobotics), 20 μM or 40 μM oligonucleotide capture probes (cf. table 1), which have been dissolved in 50% (Vol./Vol.) in DMSO, were spotted on poly-L-lysine slides (Sigma) in two subarrays. Each slide was also spotted with spotting control (5′-Cy5-tctagacagccactcata-3′) (SEQ ID NO: 55) (Cy5 labeled oligonucleotide), hybridization control (5′-gattggacgagtcaggagc-3′) (SEQ ID NO: 56) oligonucleotide with unrelated sequence referring to gyrA, whose Cy5 labeled complement oligonucleotide would be included in hybridization solution) and process control (5′-taatgggtaaataccatcc-3′) (SEQ ID NO: 57) oligonucleotide with consensus sequence of gyrA). After spotting, the slides were irradiated with UV light at 120 mJ/m² using UV crosslinker (Biometra) and blocked using an aqueous blocking solution (0.18 M succinic anhydride in methyl-pyrrolidinone/44 mM Na-borate pH 8.0) for 10 min, followed by rinse in distilled water and subsequently in 100% ethanol, and finally dried for about 10 min,

4 Amplification and Labeling

An amplification of target DNA an concurrent labeling was performed using the following primers:

Forward primer GyrA_coli_F3 (5′-acgtactaggcaatgactgg-3′) (SEQ ID NO: 58); and

reverse primer GyrA_coli_R3 (5′-agagtcgccgtcgatggaac-3′) (SEQ ID NO: 59).

The 50 μl PCR mixture included about 80 ng template (genomic DNA of E. coli), 0.4 μM (pmol/L) of each primer, 0.1 mM dATP, 0.1 mM dGTP, 0.1 mM dTTP, 0.06 mM dCTP, 0.04 mM Cy5-dCTP, 1.5 mM Mg²⁺ and 2.5 U Taq polymerase (Eppendorf). The PCRs were performed in a thermocycle (Eppendorf) using the same parameters as described before. The amplified 189 hp fragment, which was purified using QIAquick PCR purification kit (Qiagen) according to the manual provided by the manufacture, was used for hybridization.

5. Hybridization, Washing and Scanning

The purified amplicon in 40 μl hybridization solution (6×SSPE) plus 0.1 pmol Cy5 labeled DNA for the hybridization control were incubated on the slides prepared as above at 45° C. over night in a hybridization chamber (Corning) or alternatively three hours in a hybridization station. For manual hybridization 4 pmol target DNA was used, while for hybridization in a hybridization station 0.78 pmol target DNA was used. After hybridization, the slides were washed with 2×SSC, 0.1% (w/v) SDS for 15 min, with 0.2×SSC for 3 min at room temperature and dried with N₂. For detection slides were scanned using Array Scanner GMS 418 (Affymetrix) at Cy5 channel. The images were analyzed using ImaGene (BioDiscovery) and saved as plain-text file as raw data.

The results obtained by means of the array correspond to the results achieved by means of sequencing. The present method, therefore, provides an efficient means to rapidly, i.e. within about there to 5 hours, determine the presence or absence of a quinolone sensitive or resistant strain.

6. Hybridisation of Labelled Target DNA of Clinical Isolate on Microarray

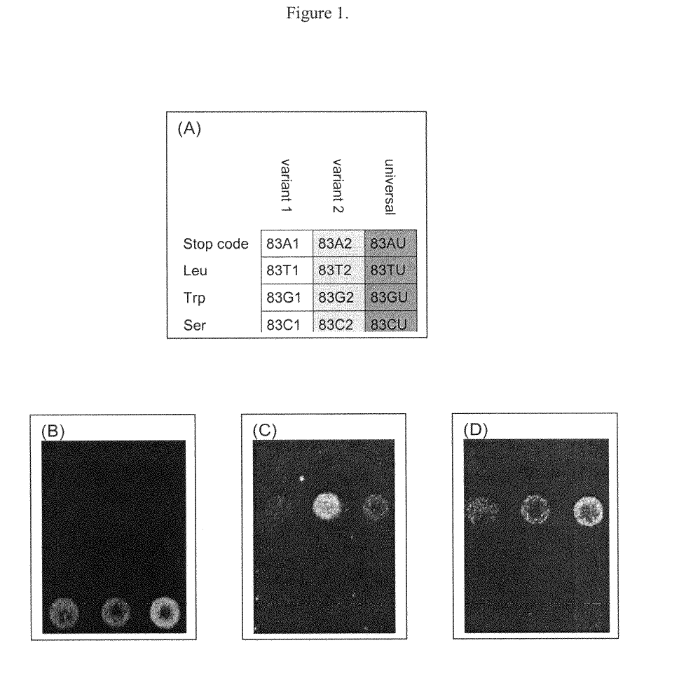

To further demonstrate the applicability of the claimed invention, a micro-array was designed to evaluate position 83. An array was prepared with the capture probes depicted in Table 1 for position 83. The layout of the probes is depicted in FIG. 1(A). Two variants were used: GTC at position 85 for variant 1, and GTT at position 85 for variant 2. The results are depicted in FIG. 1. Panel (B) depicts a quinolone-sensitive E. coli, while panels (C) and (D) show two different E. coli variants with quinolone resistance.

We claim:

1. A method for detecting the presence of quinolone resistant E. coli strains in a biological sample, comprising
(i) obtaining DNA from a biological sample;
(ii) optionally isolating DNA from the sample and/or amplifying the DNA contained in the sample with primers specific for a given target sequence;
(iii) contacting the DNA contained in the biological sample or obtained in step (ii) with a micro-array comprising at specific predetermined locations of the array two sets of capture probes, derived from the sequence of a gyrA gene of E. coli, comprising the sequence R₁—(X)—R₂, wherein
(a) X designates all permutations of the triplet at amino acid position 83 and 87 of the gyrA polypeptide of E. coli,
(b) R₁ and R₂ are sequences derived from the gyrA gene of E. coli adjacent to the triplet of either position 83 or 87 of the gyrA polypeptide and comprising of from about 5 to 20 nucleotides each;
under conditions allowing hybridization of complementary strands; and
(iv) determining at which location on the array binding occurs,
wherein a change in the DNA at least one of said positions results in a change of an amino acid and is indicative of the development of a resistance against quinolones,
further wherein the micro-array additionally comprises at specific predetermined locations of the array at least one additional set of capture probes, derived from the sequence of a parC gene of E. coli, and selected from a nucleotide sequence comprising the sequence R₃—(Y)—, wherein
(a) Y designates all permutations of the triplet at amino acid position 80 or 84 of the parC polypeptide of E. coli;
(b) R₃ and R₄ are sequences derived from the parC gene of E. coli adjacent to the triplet of either position 83 or 84 of the parC polypeptide and comprising of from about 5 to 20 nucleotides each;
wherein a change in the nucleic acid at least one position in the sequence results in a change of an amino acid and is indicative of the development of a resistance against quinolones.

2. The method according to claim 1, wherein the DNA obtained from a biological sample is amplified by means of PCR.

3. The method according to claim 2, wherein the DNA is fragmented prior to the contacting step.

4. The method according to claim 3, wherein the DNA is fragmented to pieces having a length of from about 10 to about 40 nucleotides.

5. The method according to claim 1, wherein the micro-array contains the capture-probes listed in table I.

6. The method according to claim 1 wherein the DNA is labeled prior to contacting it with the capture probes.

7. The method according to claim 6, wherein the label is selected from the group consisting of fluorescence label, calorimetric label, radioactive label, and an enzymatically detectable label.

8. A micro-array containing at specific predetermined locations of the array two sets of capture probes, derived from the sequence of a gyrA gene of E. coli, comprising the sequence R₁—(X)—R₂, wherein (a) X designates all permutations of the triplet at amino acid position 83 and 87 of the gyrA polypeptide of E. coli and (b) R₁ and R₂ are sequences derived from the gyrA gene of E. coli adjacent to the triplet of either position 83 or 87 of the gyrA polypeptide and comprising of from about 5 to 20 nucleotides.

9. The micro-array according to claim 8, further comprising at specific predetermined locations of the array at least one additional set of capture probes selected from a nucleotide sequence derived from the sequence of a parC gene of E. coli, and comprising the sequence R₃—(Y)—R₄, wherein (a) Y designates all permutations of the triplet at amino acid position 80 or 84 of the parC polypeptide of E. coli and (b) R₃ and R₄ are sequences derived from the parC gene of E. coli adjacent to the triplet of either position 83 or 84 of the parC polypeptide and comprising of from about 5 to 20 nucleotides.

10. A kit for detecting the presence of a quinolone resistant E. coli strain in a biological sample, containing a micro-array according to claim 11 and optionally buffers and reagents.
